(19) United States
(12) Patent Application Publication
(10) Pub. No.: US 2026/0283465 A1
(43) Pub. Date: Sep. 24, 2026

(54) Sensor system and computer-implemented method for evaluating a physiology of a post-infarction patient

(71) Applicant: BIOTRONIK SE & Co. KG, Berlin (DE)

(72) Inventors: Thomas DOERR, Berlin (DE); Thomas KRUCZYK, Berlin (DE); Sebastian SCHULZE-LUCKOW, Berlin (DE); Frank BECKER, Berlin (DE)

(73) Assignee: BIOTRONIK SE & Co. KG, Berlin (DE)

(21) Appl. No.: 19/474,297

(22) PCT Filed: Apr. 10, 2024
(86) PCT No.: PCT/EP2024/059711

§ 371 Date: Oct. 10, 2025

(30) Foreign Application Priority Data
Apr. 25, 2023 (EP) 23169643.6

(51) Int. Cl. A61B 5/00; A61B 5/145; A61B 5/29; A61B 5/329; A61B 5/333; A61B 5/352; A61B 5/355; A61B 5/358

(52) U.S. Cl. CPC A61B 5/0006; A61B 5/14542; A61B 5/29; A61B 5/329; A61B 5/333; A61B 5/352; A61B 5/355; A61B 5/358; A61B 5/686; A61B 2560/0214; A61B 2560/0468

(57) ABSTRACT

A sensor system including a leadless sensor device for implantation under the skin or for attachment to the skin of a patient for evaluating a physiology of a post-infarction patient, the leadless sensor device including a power source, at least one sensing unit for recording at least one ECG lead, a communication interface for direct or indirect communication of the ECG data to a data acquisition system external to the patient body, and an evaluation unit arranged in the leadless sensor device and/or in the data acquisition system, wherein the evaluation unit is configured to detect a Q-wave in the ECG lead and to analyze pathological indicators of the Q-wave. Furthermore, a computer-implemented method for evaluating a physiology of a post-infarction patient is also disclosed.

### CROSS-REFERENCE TO RELATED APPLICATIONS

This application is the United States National Phase under 35 U.S.C. § 371 of PCT International Patent Application No. PCT/EP 2024/059711, filed on Apr. 10, 2024, which claims the benefit of European Patent Application No. 23169643.6, filed on Apr. 25, 2023, the disclosures of which are hereby incorporated by reference herein in their entireties.

### TECHNICAL FIELD

The invention relates to a sensor system for implantation under the skin or for attachment to the skin of a patient for evaluating a physiology of a post-infarction patient.

Furthermore, the invention relates to a computer-implemented method for evaluating a physiology of a post-infarction patient.

### BACKGROUND

Follow up of post-infarction patients usually occurs over a period of several weeks or months. In particular, the diagnosis of post-infarction patients to date is both personnel-and cost-intensive. Individual adaptation of therapy and rehabilitation must be accompanied by frequent visits to a healthcare provider. Since this is time-consuming for the patients, these checks do not take place often enough and the therapy/rehabilitation is not optimally controlled, thus worsening a patients' prognosis.

The present disclosure is directed toward overcoming one or more of the above-mentioned problems, though not necessarily limited to embodiments that do.

### SUMMARY

It is therefore an object of the present invention to provide an improved sensor system and method for evaluating a physiology of a post-infarction patient capable of providing an optimal control of a patient therapy/rehabilitation.

The object is solved by a sensor system comprising a leadless sensor device for implantation under the skin or for attachment to the skin of a patient for evaluating a physiology of a post-infarction patient having the features of claim 1.

Furthermore, the object is solved by a computer-implemented method for evaluating a physiology of a post-infarction patient having the features of claim 15.

Further developments and advantageous embodiments are defined in the dependent claims.

The present invention provides a sensor system, in particular a leadless sensor system, comprising a leadless sensor device for implantation under the skin or for attachment to the skin of a patient for evaluating a physiology of a post-infarction patient. The leadless sensor device comprises a power source and at least one sensing unit for recording at least one lead of an electrocardiogram (ECG), also called ECG lead. An ECG lead is a graphical description of the electrical activity of the heart.

In addition, the leadless sensor device comprises a communication interface for direct or indirect communication of the ECG data to a data acquisition system external to the patient body, and an evaluation unit arranged in the leadless sensor device and/or in the data acquisition system, wherein the evaluation unit is configured to detect a Q-wave in the ECG lead and to analyze pathological indicators of the Q-wave.

Furthermore, the present invention provides a computer-implemented method for evaluating a physiology of a post-infarction patient. The method comprises providing a sensor system, in particular a leadless sensor system, comprising a leadless sensor device, the leadless sensor device comprising a power source and recording at least one ECG lead by means of at least one sensing unit.

Moreover, the method comprises providing direct or indirect communication of the ECG data to a data acquisition system external to the patient body by means of a communication interface, and detecting a Q-wave in the ECG lead and analyzing pathological indicators of the Q-wave by means of an evaluation unit arranged in the leadless sensor device and/or in the data acquisition system.

The Q-wave is the first wave of the QRS-complex in the electrocardiogram. It points in the opposite direction of the R-wave.

The Q-wave results from retrograde excitation propagation, i.e., propagation from the apex to the base of the heart, which occurs primarily in the papillary muscles and septum interventriculare.

The Q-waveform usually occurs only in leads with high R-waveforms, for example, lead I and aVL in a left type or lead II and III, and aVF in a right type or a steep type. It usually has a width of up to 0.03 seconds and a depth of about 0.2-0.3 mV. However, in aVR and Nehb D, its amplitude can be much more pronounced.

The widened and deepened Q-wave is considered a pathological change. It is referred to as pathologic Q or Pardee-Q. Pathologic Q occurs when the width of the Q-wave is greater than 0.04 seconds and its depth is greater than ¼ of the highest R-wave.

Pathologic Q occurs in the limb leads in the following situations, for example a pronounced anterior wall infarction, a posterior wall infarction and/or a heart wall aneurysm.

Whether an infarct is fresh or old cannot be deduced from the Q-waveform alone. For this purpose, disturbances in excitation regression must also be assessed. Pathologic Q in the chest wall leads is seen in, among others septal infarction or septal hypertrophy.

An idea of the present invention is thus to allow telemedical follow-up of post-infarction patients by recording and analyzing the myocardial recovery and transmitting it telemedically.

According to an aspect of the invention, the leadless sensor device is configured to be implanted under the skin or attached to the skin of the patient in such a way that an individual ECG, in which at least one infarct-related, pathological indicator of the Q-wave, in particular a Q-wave change, is present and/or expected, is detectable.

Thus, the Q-wave in the ECG lead can be detected and pathological indicators of the Q-wave can be analyzed by the evaluation unit.

According to a further aspect of the invention, the evaluation unit is configured to analyze a width of the Q-wave, to evaluate a T-wave, a ST-segment and/or to analyze an amplitude of the Q-wave in relation to an amplitude of an R-wave of a same QRS complex.

This enables an enhanced a multi-dimensional analysis of the pathological indicators of the Q-wave taking into account features of the Q-wave itself as well as further features related to the Q-wave and/or features that can be used as a reference to compare the Q-wave data against.

According to a further aspect of the invention, the leadless sensor device comprises a memory unit configured to record Q-wave data between interrogations and/or transmissions of the leadless sensor device. The recorded Q-wave data can thus be sent to the evaluation unit for further analysis.

According to a further aspect of the invention, an evaluation and/or the transmission of the Q-wave data is performed at predetermined intervals, in particular hourly, daily and/or weekly intervals.

The evaluation and/or transmission of the recorded Q-wave data thus enables a close follow-up of the patient, wherein a therapy can be designed and/or adapted in line with analysis results of the recorded Q-wave data.

According to a further aspect of the invention, the leadless sensor device is configured to record at least one parameter representing a physical load on the body of the patient.

Such a parameter can be an acceleration rate of the patient body recorded by the leadless sensor device. This parameter can be used as a further input to the evaluation unit which enables a determination whether or not external factors such as a physical load on the body of the patient influence the Q-wave data.

According to a further aspect of the invention, the evaluation unit is configured to evaluate the Q-wave data when the physical load on the body of the patient exceeds a predetermined threshold value.

In this way, if the physical load on the body of the patient is found to have an effect on the patient physiology, said data is taken into consideration when evaluating the Q-wave data.

According to a further aspect of the invention, the communication interface is configured to communicate with the data acquisition system via a coil communication, a galvanic communication method, RF-telemetry in a frequency range between 500 MHz and 1 GHz, RF telemetry in a frequency range above 1 GHz and/or via an additional leadless sensor device configured to act as a relay station.

The communication interface thus provides a wide range of communication methods hence offering a broad compatibility with the data acquisition systems.

According to a further aspect of the invention, the power source of the leadless sensor device is configured to provide a run time of the leadless sensor device of between three months and one year. This time frame advantageously covers a patient follow-up period. Thus, the power source and/or power usage of the leadless sensor device can be adapted to the intended patient follow-up period.

According to a further aspect of the invention, the evaluation unit is configured to evaluate the Q-wave morphology using a machine learning algorithm. The machine learning algorithm advantageously enables to identify non-linear correlations of the Q-wave data to pathological changes in the patient physiology.

According to a further aspect of the invention, if a recovery or non-recovery of the patient physiology is detected, the leadless sensor device is configured to be switched from a first operating mode to a second operating mode, in particular a message mode for third party devices and/or an active or passive monitoring mode.

A change or non-change in patient physiology is an important indicator whether or not patient recovery is in line with expectations and/or therapy goals.

According to a further aspect of the invention, the leadless sensor device is configured to determine if a detection is identical in different operating modes comprising a sitting mode, a standing mode, a running mode and/or a walking mode and/or using information on whether medication was administered during a predetermined interval prior to the detection.

Thus, a patient reaction to medical intake can be monitored by detecting changes in patient physiology. Furthermore, the influence of external factors such as physical exercise on patient physiology can be taken into account when evaluating ECG data.

According to a further aspect of the invention, the leadless sensor device is configured to connect to a wearable device comprising an oxygen sensor to determine if the detected recovery or non-recovery of the patient physiology is due to a blood oxygen level being below a predetermined threshold value and/or wherein the leadless sensor device is configured to calculate a probability of a recurrent or new infarct. Thus, the effect of the blood oxygen level on patient physiology is taken into account when evaluating said ECG data.

According to a further aspect of the invention, the leadless sensor device is configured to classify a condition of the patient physiology, wherein depending on a classification result, the leadless sensor device is set into an associated operating mode.

The operating mode can be a messaging mode informing the patient and/or healthcare provider of the data analysis and/or providing a recommended action to the patient such as a medication intake, a physical activity and/or scheduling an appointment with the healthcare provider in charge of patient follow-up.

The herein described features of the leadless sensor device for implantation under the skin or for attachment to the skin of a patient for evaluating a physiology of a post-infarction patient are also disclosed for the computer-implemented method for evaluating a physiology of a post-infarction patient and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a diagram of a sensor system for evaluating a physiology of a post-infarction patient according to a preferred embodiment of the invention; and

FIG. 2 shows a flowchart of a computer-implemented method for evaluating a physiology of a post-infarction patient according to the preferred embodiment of the invention; and

FIG. 3 shows a progression of a Q waveform in a myocardial infarction-follow-up patient according to the preferred embodiment of the invention.

### DETAILED DESCRIPTION

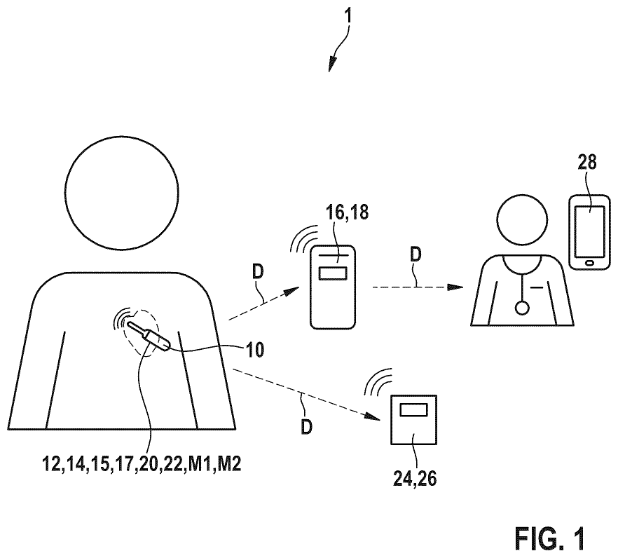

The (leadless) sensor system 1 shown in FIG. 1 comprises a leadless sensor device 10 and a data acquisition system 16 external to the patient body.

The leadless sensor device 10 for implantation under the skin or for attachment to the skin of a patient for evaluating a physiology of a post-infarction patient shown in FIG. 1 comprises a power source 12 and at least one sensing unit 14 for recording at least one ECG lead 15.

In addition, the leadless sensor device 10 comprises a communication interface 17 for direct or indirect communication of the ECG data to the data acquisition system 16 external to the patient body and an evaluation unit 18 arranged in the data acquisition system 16, wherein the evaluation unit 18 is configured to detect a Q-wave in the ECG lead 15 and to analyze pathological indicators of the Q-wave.

Alternatively, the evaluation unit 18 can be arranged in the leadless sensor device 10 or in both the data acquisition system 16 and the leadless sensor device 10.

The sensor system 1 of FIG. 1 for telemedical monitoring further comprises a patient device. A patient wears a leadless sensor device 10, in particular a subcutaneously implantable sensor, whose implantation location depends on the affected infarct area of the heart. The leadless sensor device 10 transmits Q-wave information to a remote physician follow-up system via a patient device.

A preferred implementation of the Q-wave information is to determine the amplitude ratio of the Q-wave and R-wave in predetermined intervals, e.g. daily. Such information can be presented as a trend and used for follow-up.

The leadless sensor device 10 is configured to be implanted under the skin or attached to the skin of the patient in such a way that an individual ECG, in which at least one infarct-related, pathological indicator of the Q-wave, in particular a Q-wave change, is present and/or expected, is detectable.

Furthermore, the evaluation unit 18 is configured to analyze a width of the Q-wave, to evaluate a T-wave, a ST-segment and/or to analyze an amplitude of the Q-wave in relation to an amplitude of an R-wave of a same QRS complex.

The leadless sensor device 10 moreover comprises a memory unit 20 configured to record Q-wave data D between interrogations and/or transmissions of the leadless sensor device 10.

An evaluation and/or the transmission of the Q-wave data D is performed at predetermined intervals, in particular hourly, daily and/or weekly intervals. In addition, the leadless sensor device 10 is configured to record at least one parameter 22 representing a physical load on the body of the patient.

The evaluation unit 18 is configured to evaluate the Q-wave data D when the physical load on the body of the patient exceeds a predetermined threshold value. The communication interface 17 is configured to communicate with the data acquisition system 16 via a coil communication, a galvanic communication method, RF-telemetry in a frequency range between 500 MHz and 1 GHz, RF telemetry in a frequency range above 1 GHz and/or via an additional leadless sensor device 10 configured to act as a relay station.

The data acquisition system 16 is further configured to transmit the Q-wave data D, an evaluation result of the Q-wave data D and/or a message triggered by the data acquisition system 16 or the evaluation unit 18 in response to the evaluation of the Q-wave data D to a remote follow-up system 28, in particular a follow-up system of a healthcare provider.

The power source 12 of the leadless sensor device 10 is configured to provide a run time of the leadless sensor device 10 of between three months and one year. Furthermore, the evaluation unit 18 is configured to evaluate the Q-wave morphology using a machine learning algorithm.

The machine learning algorithm is configured to learn across patients or on a patient-by-patient basis.

The machine learning algorithm is trained using labeled ground truth data as well as said Q-wave data D obtained by the leadless sensor device 10. The model can further process oxygen/respiratory data and/or parameters representing a load on the patient body. The machine learning algorithm is further configured to identify a patient group to which the patient belongs according to a QRS-data history and which measures are sensibly initiated for said group.

If a recovery or non-recovery of the patient physiology is detected, the leadless sensor device 10 is configured to be switched from a first operating mode M1 to a second operating mode M2, in particular a message mode for third party devices and/or an active or passive monitoring mode.

The leadless sensor device 10 is further configured to determine if a detection is identical in different operating modes comprising a sitting mode, a standing mode, a running mode and/or a walking mode and/or using information on whether medication was administered during a predetermined interval prior to the detection.

In addition, the leadless sensor device 10 is configured to connect to a wearable device 24 comprising an oxygen sensor 26 to determine if the detected recovery or non-recovery of the patient physiology is due to a blood oxygen level being below a predetermined threshold value and/or wherein the leadless sensor device 10 is configured to calculate a probability of a recurrent or new infarct.

Possible conditions are e.g., a non-recovery of the patient physiology is detected and the blood oxygen level being is below the predetermined threshold value. This would indicate a critical patient condition. Alternatively, e.g., a non-recovery of the patient physiology is detected and the blood oxygen level is above the predetermined threshold value. In this case further observation of the patient physiology would be advisable.

Further alternatively, if a recovery of the patient physiology is detected and the blood oxygen level being is below the predetermined threshold value, a measurement error could be at cause. In addition, if a recovery of the patient physiology is detected and the blood oxygen level is above the predetermined threshold value, an optimal therapy outcome is achieved. Depending on the above categories, a different operating mode of the leadless sensor device 10 is set.

Moreover, the leadless sensor device 10 is configured to classify a condition of the patient physiology, wherein depending on a classification result, the leadless sensor device 10 is set into an associated operating mode.

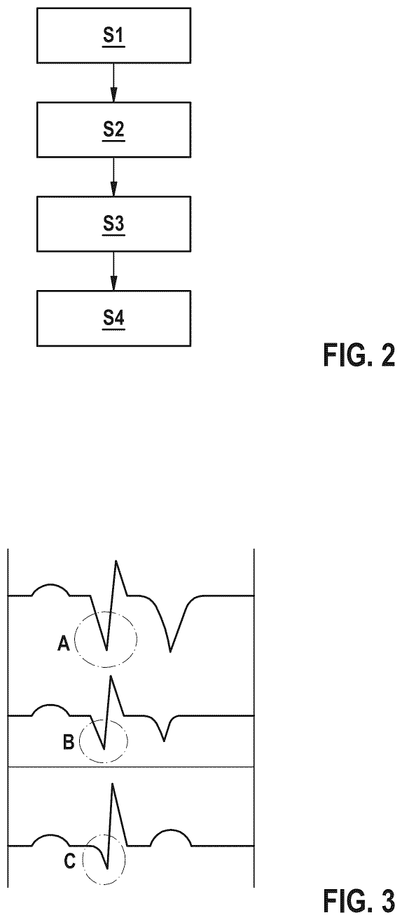

FIG. 2 shows a flowchart of a computer-implemented method for evaluating a physiology of a post-infarction patient according to the preferred embodiment of the invention.

The method for evaluating a physiology of a post-infarction patient comprises providing a sensor system 1 comprising a leadless sensor device 10, the leadless sensor device 10 comprising a power source 12 and recording at least one ECG lead 15 by means of at least one sensing unit 14.

Moreover, the method comprises providing direct or indirect communication of the ECG data to a data acquisition system 16 external to the patient body by means of a communication interface 17, and detecting a Q-wave in the ECG lead 15 and analyzing pathological indicators of the Q-wave by means of an evaluation unit 18 arranged in the leadless sensor device 10 and/or in the data acquisition system 16.

The evaluation of pathological Q and its regression can provide an indication of the recovery of a post-myocardial infarction patient and can therefore be used for follow-up.

FIG. 3 shows the progression of the Q waveform in a patient who is progressively recovering from a myocardial infarction. In the chronic stage of the infarction, a pronounced Q-wave A is still evident at first, which is larger in amplitude than the following R-wave. Over time, this pathological Q-wave decreases in size B and subsequently normalizes C.

Therefore, for monitoring the post-myocardial infarction patient, the amplitude of the Q-wave in relation to the R-wave amplitude should preferably be determined and evaluated e.g., as a trend function.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### Reference Signs

1 (leadless) sensor system
10 leadless sensor device
12 power source
14 sensing unit
15 ECG lead
16 data acquisition system
17 communication interface
18 evaluation unit
20 memory unit
22 parameter
24 wearable device
26 oxygen sensor
28 remote follow-up system
D Q-wave data
M1 first operating mode
M2 second operating mode
S1-S4 method steps

1. Sensor system comprising a leadless sensor device for implantation under the skin or for attachment to the skin of a patient for evaluating a physiology of a post-infarction patient, the leadless sensor device comprising a power source;
at least one sensing unit for recording at least one ECG lead; a communication interface for direct or indirect communication of the ECG data to a data acquisition system external to the patient body; and
an evaluation unit arranged in the leadless sensor device and/or in the data acquisition system, wherein the evaluation unit is configured to detect a Q-wave in the ECG lead and to analyze pathological indicators of the Q-wave.

2. Sensor system of claim 1, wherein the leadless sensor device is configured to be implanted under the skin or attached to the skin of the patient in such a way that an individual ECG, in which at least one infarct-related, pathological indicator of the Q-wave, in particular a Q-wave change, is present and/or expected, is detectable.

3. Sensor system of claim 1, wherein the evaluation unit is configured to analyze a width of the Q-wave, to evaluate a T-wave, a ST-segment and/or to analyze an amplitude of the Q-wave in relation to an amplitude of an R-wave of a same QRS complex.

4. Sensor system of claim 1, wherein the leadless sensor device comprises a memory unit configured to record Q-wave data (D) between interrogations and/or transmissions of the leadless sensor device.

5. Sensor system of claim 1, wherein an evaluation and/or the transmission of the Q-wave data (D) is performed at predetermined intervals, in particular hourly, daily and/or weekly intervals.

6. Sensor system claim 1, wherein the leadless sensor device (10) is configured to record at least one parameter representing a physical load on the body of the patient.

7. Sensor system of claim 6, wherein the evaluation unit is configured to evaluate the Q-wave data (D) when the physical load on the body of the patient exceeds a predetermined threshold value.

8. Sensor system of claim 1, wherein the communication interface is configured to communicate with the data acquisition system via a coil communication, a galvanic communication method, RF-telemetry in a frequency range between 500 MHz and 1 GHz, RF telemetry in a frequency range above 1 GHz and/or via an additional leadless sensor device configured to act as a relay station.

9. Sensor system of claim 1, wherein the power source of the leadless sensor device is configured to provide a run time of the leadless sensor device of between three months and one year.

10. Sensor system of claim 1, wherein the evaluation unit is configured to evaluate the Q-wave morphology using a machine learning algorithm.

11. Sensor system of claim 1, wherein if a recovery or non-recovery of the patient physiology is detected, the leadless sensor device is configured to be switched from a first operating mode to a second operating mode, in particular a message mode for third party devices and/or an active or passive monitoring mode.

12. Sensor system of claim 1, wherein the leadless sensor device is configured to determine if a detection is identical in different operating modes comprising a sitting mode, a standing mode, a running mode and/or a walking mode and/or using information on whether medication was administered during a predetermined interval prior to the detection.

13. Sensor system of claim 11, wherein the leadless sensor device is configured to connect to a wearable device comprising an oxygen sensor to determine if the detected recovery or non-recovery of the patient physiology is due to a blood oxygen level being below a predetermined threshold value and/or wherein the leadless sensor device is configured to calculate a probability of a recurrent or new infarct.

14. Sensor system of claim 13, wherein the leadless sensor device-is configured to classify a condition of the patient physiology, wherein depending on a classification result, the leadless sensor device is set into an associated operating mode.

15. Computer-implemented method for evaluating a physiology of a post-infarction patient, comprising the steps of:
providing a sensor system comprising a leadless sensor device, the leadless sensor device comprising a power source;
recording at least one ECG lead by means of at least one sensing unit;
providing direct or indirect communication of the ECG data to a data acquisition system external to the patient body by means of a communication interface; and
detecting a Q-wave in the ECG lead and analyzing pathological indicators of the Q-wave by means of an evaluation unit arranged in the leadless sensor device and/or in the data acquisition system.
